(19) United States
(12) Patent Application Publication
(10) Pub. No.: US 2013/0072968 A1
(43) Pub. Date: Mar. 21, 2013

(54) Reinforced Suture Strip and Methods of Use

(71) Applicants: Robert L. McClure, JR., Elizabethton, TN (US); Kellie Cunningham, Knoxville, TN (US); Randel B. Holmes, Knoxville, TN (US)

(72) Inventors: Robert L. McClure, JR., Elizabethton, TN (US); Kellie Cunningham, Knoxville, TN (US); Randel B. Holmes, Knoxville, TN (US)

(73) Assignee: WEBTEC Converting, LLC., Knoxville, TN (US)

(21) Appl. No.: 13/234,287

(22) Filed: Sep. 16, 2011

(51) Int. Cl. A61B 17/03

(52) U.S. Cl. 606/215

(57) ABSTRACT

A reinforced suture strip that incorporates resilient bands oriented parallel to one another along a longitudinal axis of the suture strip. Each band includes a semirigid strip of resilient material, said strip generally extending substantially the length of the suture strip, substantially parallel to the longitudinal axis of the suture strip. The reinforced suture strip inhibits or minimizes both abductive and transverse movement by the opposing sides of flesh about an incisive wound. The present invention also comprises in some of its embodiments methods and processes for treating wounds using reinforced suture strips as described herein.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of Invention The present invention relates generally to methods and devices used to close a wound or to tie bind or constrict a portion of a body and in particular to the use of suture strips to prevent suture stretching, encourage the formation of fibrous cross-linkages, and reduce widening of an incision, thereby inhibiting scar formation, reopening of a wound, and infection.

2. Description of the Related Art

In order for incisions or incision-based wounds to heal, it is frequently necessary to immobilize the areas of skin on opposing sides of an incision or incision-based wound with respect to the incision or incision-based wound, so that abutting areas of skin tissue on opposing sides of the incisive wound do not slide or separate with respect to each other. Hereinafter, “incisive wounds” is understood to encompass a wide variety of incisions breaking the surface of the skin or epidermis, including but not limited to accidental cuts, surgical incisions, and other lacerations.

Further, it is generally desirable to prevent or inhibit an incisive wound from reopening during the healing process. Reopening of the wound delays the healing process and also increases the likelihood for the appearance of substantial scar tissue and later visible scarring. Additionally, an open wound is at a heightened risk of infection throughout the healing process. In particular, microorganisms such as bacteria and fungi will attempt to establish themselves in the moisture of the exudate extruded from the wound during the healing process.

Scarring is a natural part of the healing process. Scar tissue consists mainly of protein collagen formed during the skin's process of wound repair. With the exception of very minor lesions, skin wounds following accidents, disease or surgery all result in some degree of visible scarring. Where the scar tissue is large or in a prominent position on the body, it can be readily apparent to a casual observer and embarrassing or otherwise troubling for the scarred person.

It is therefore desirable to have a device, or a method, to substantially immobilize the two areas of skin tissue on opposing sides of an incisive wound, in order at least to keep abutting skin tissue on opposing sides of the incisive wound from sliding or separating with respect to each other, to facilitate the formation of linkages between areas of skin tissue on opposing sides of the incisive wound, thereby hastening the healing process and helping to minimize the development of scar tissue and the appearance of scars, and to prevent the incisive wound from reopening during the healing process.

### BRIEF SUMMARY OF THE INVENTION

The present invention in some of its embodiments is directed toward a reinforced suture strip that incorporates resilient bands oriented parallel to one another along a longitudinal axis of the suture strip. In some of its embodiments, the reinforced suture strip inhibits or minimizes both abductive and transverse movement by the opposing sides of flesh about an incisive wound. The present invention also comprises in some of its embodiments methods and processes for treating wounds using reinforced suture strips as described herein. Methods and devices used to close a wound include in particular the use of reinforced suture strips to prevent suture stretching, encourage the formation of fibrous cross-linkages, and reduce widening of an incision, thereby inhibiting scar formation, reopening of a wound, and infection.

In some example embodiments, a reinforced suture strip according to the present invention comprises a bottom layer fabricated from a nonwoven, spunlaced polyester fabric; a top layer a nonwoven, breathable polymer fabric; and at least two parallel band members positioned between the top layer and the bottom layer. Each band member includes a semirigid strip of resilient material, said strip generally extending substantially the length of the suture strip. Each band member extends along the length of the suture strip substantially parallel to the longitudinal axis of the suture strip.

By substantially immobilizing the two areas of skin tissue on opposing sides of an incisive wound, the reinforced suture strip helps to keep abutting skin tissue on opposing sides of the incisive wound from sliding or separating with respect to each other. By substantially immobilizing the two areas of skin tissue on opposing sides of an incisive wound, the reinforced suture strip helps to prevent suture stretching and helps to prevent any widening of an incision of reopening of a wound that has begin to close. Preventing reopening of the wound helps to avoid infection of the wound or the entry of foreign biological and non-biological entities. Further, by substantially immobilizing the skin tissue around an incisive wound, the reinforced suture strip facilitates the formation of linkages between areas of skin tissue on opposing sides of the incisive wound, hastening the healing process and helping to minimize the development of scar tissue and the appearance of scars.

Some embodiments of the present invention include methods and processes in which a reinforced suture strip is combined with an angled incision or angled incisive wound so as to reduce the appearance of scars from incisions. In some circumstances, such as surgery, it is necessary for a medical practitioner to make an incision in the patient's skin. If the incision made is a vertical incision—i.e., the main direction of the incision is substantially perpendicular to the plane of the patient's top-most layer of skin—then, during the healing process, the areas of skin on opposing sides of the incision must form fibrous cross-linkages in a largely horizontal direction—that is, substantially parallel to the top-most layer of skin. Because the separated areas of skin on opposing sides of the incision will also likely be pulled and moved in a largely horizontal direction during the healing process, the body's healing process must work almost directly against the expected movement of the separated areas of skin. On the other hand, if the incision is made angled—that is, the angle of incision is substantially acute with respect to the plane of the top-most layer of skin—then, during the healing process, the areas of skin on opposing sides of the incision will form fibrous cross-linkages in substantially angled and vertical directions, without directly competing with the horizontal movement of the separated opposing areas of skin on either side of the incision. A reinforced suture strip further enhances and supplements the beneficial aspects of the angled incision or angled incisive wound. By substantially immobilizing the two areas of skin tissue on opposing sides of the angled incisive wound, the reinforced suture strip helps to keep abutting skin tissue on opposing sides of the angled incisive wound from sliding or separating with respect to each other. In some embodiments, the reinforced suture strip is used to supplement the wound-closing action of a conventional suture after the wound has been sutured.

### BRIEF DESCRIPTION OF THE SEVERAL VIEWS OF THE DRAWINGS

The above-mentioned and additional features of the invention will become more clearly understood from the following detailed description of the invention read together with the drawings in which:

FIG. 1 is a perspective view of an example embodiment of the present invention;

FIG. 2 is an exploded view of the example embodiment shown in FIG. 1;

FIG. 3 is a top-down view of the example embodiment shown in FIGS. 1 and 2;

FIG. 4 is a view of an example embodiment of the present invention, showing several reinforced suture strips in use on an incisive wound on a human arm;

FIG. 5 is a sectional view of the embodiment shown in FIG. 4;

FIG. 6 is a sectional view of one example embodiment of the present invention, showing a reinforced suture strip used in conjunction with a conventional suture; and

FIG. 7 is a view of an example embodiment of the present invention, showing several partially overlapping reinforced suture strips in use on an incisive wound on a human arm.

### DETAILED DESCRIPTION OF THE INVENTION

The present invention in some of its embodiments is directed toward a reinforced suture strip that incorporates resilient bands oriented parallel to one another along a longitudinal axis of the suture strip. In some of its embodiments, the reinforced suture strip inhibits or minimizes both abductive and transverse movement by the opposing sides of flesh about an incisive wound. The present invention also comprises in some of its embodiments methods and processes for treating wounds using reinforced suture strips as described herein.

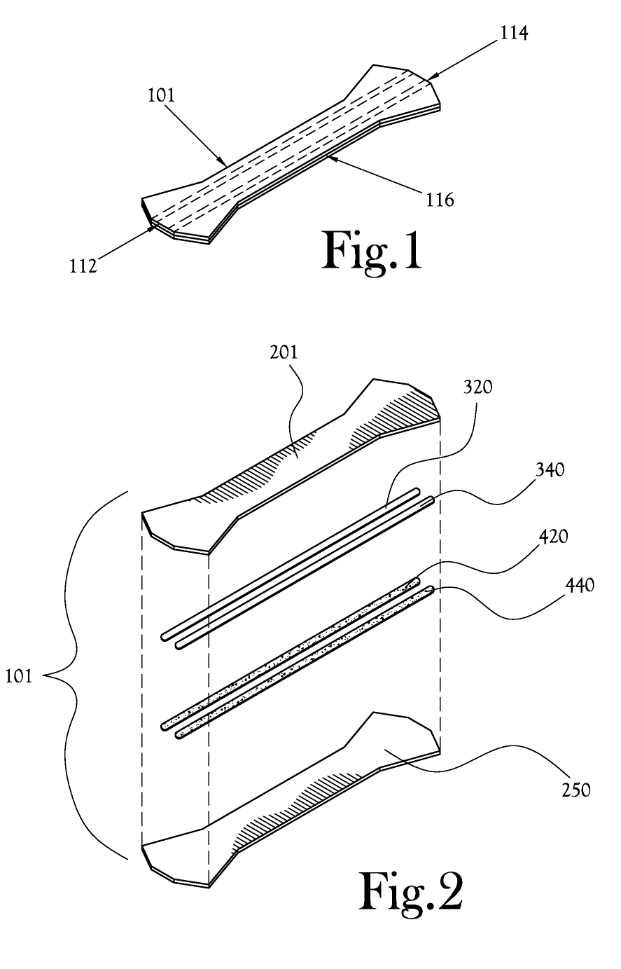

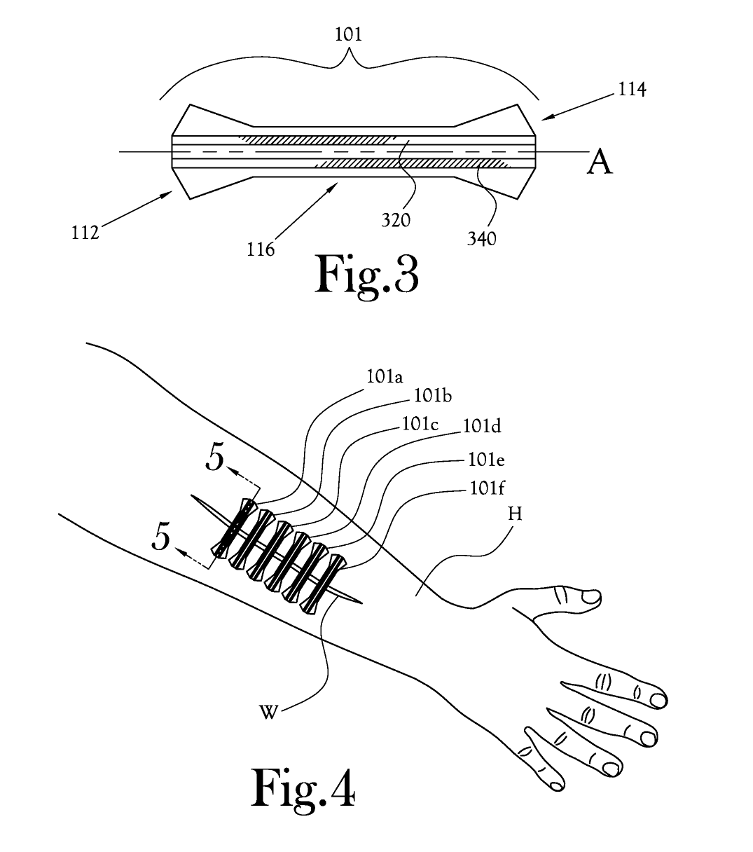

FIG. 1 is a perspective view of one example embodiment of a reinforced suture strip according to the present invention. As shown in FIG. 1, a suture strip 101 includes a length of material comprising a two end regions 112 and 114 and a middle region 116; generally, the middle region 116 is narrower at its narrowest point than the width of the two end regions 112 and 114 at their widest points. The reinforced suture strip 101 is applied to the patient's skin about the incision so that each end region 112 and 114 is positioned on an opposing side of the incision from the other end region. As shown in FIG. 1 and in the exploded view of the same example embodiment in FIG. 2, the suture strip 101 includes a top layer 201 and a bottom layer 251. In the illustrated example embodiment, between the top layer 201 and the bottom layer 251 are two band members 320 and 340. Each band member 320 and 340 includes a semi-rigid strip of resilient material, said strip generally extending substantially the length of the suture strip 101. As shown in the top-down view of FIG. 3, each band member 320 and 340 extends along the length of the suture strip 101, substantially parallel to the longitudinal axis of the suture strip 101, indicated by the dashed line A. In the example embodiment shown in FIGS. 1 through 3, two band members are used. However, in various embodiments, a different number of band members are used.

In several embodiments, the bottom layer 251 generally is the layer applied to a patient's skin and wound, as described below. In some embodiments, the bottom layer 251 generally is fabricated from a polymer material that will minimize irritation to the patient's skin and will allow the patient's skin to breathe. In some embodiments, the bottom layer 251 is fabricated from a polyester fabric. In some embodiments, the bottom layer 251 is fabricated from a nonwoven, spunlaced polyester fabric, such as SONTARA®, available from E.I. DuPont Nemours & Co. (Wilmington, Del.).

In some embodiments, the top layer 201 is fabricated from a polymer material that will allow the patient's skin to breathe. In some embodiments, the top layer 201 is fabricated from a nonwoven polymer material. In some embodiments, the top layer 201 is fabricated from a nonwoven, breathable polymer fabric, such as 3M 1533, available from Minnesota Mining & Manufacturing, Inc. (3M) (St. Paul, Minn.).

In some embodiments, the band members 320 and 340 are fabricated from a plastic material. In some embodiments, the band members 320 and 340 are fabricated from a polyester material. In some embodiments, the band members 320 and 340 are secured to the bottom layer 251 by strips of adhesive interface material 420 and 440, which are positioned between the band members 320 and 340 and the bottom layer 251, as shown in the exploded view of FIG. 2. In some embodiments, the strips of adhesive interface material 420 and 440 are fabricated from a pressure-sensitive acrylic adhesive material. In some embodiments, the strips of adhesive interface material 420 and 440 are fabricated from a pressure-sensitive, acrylic, bio-compatible adhesive material, such as 3M 1509 or 3M 9952, both available from Minnesota Mining & Manufacturing, Inc. (3M) (St. Paul, Minn.).

In one particular example embodiment, a reinforced suture strip according to the present invention comprises a bottom layer fabricated from a nonwoven, spunlaced polyester fabric; a top layer fabricated from a nonwoven, breathable polymer fabric; and two parallel band members positioned between the top layer and the bottom layer. The top layer and the bottom layer are coterminous and are approximately 2.125 inches long at the longest point. At its widest points, near each end of the suture strip, the suture strip is approximately between 0.691 inches and 0.800 inches wide. In the middle of the suture strip, at its narrowest portion, the suture strip is approximately between 0.350 inches and 0.400 inches wide. Each band member is slightly less than 2.125 inches long and is approximately between 0.080 inches and 0.115 inches wide and approximately 0.010 inches thick.

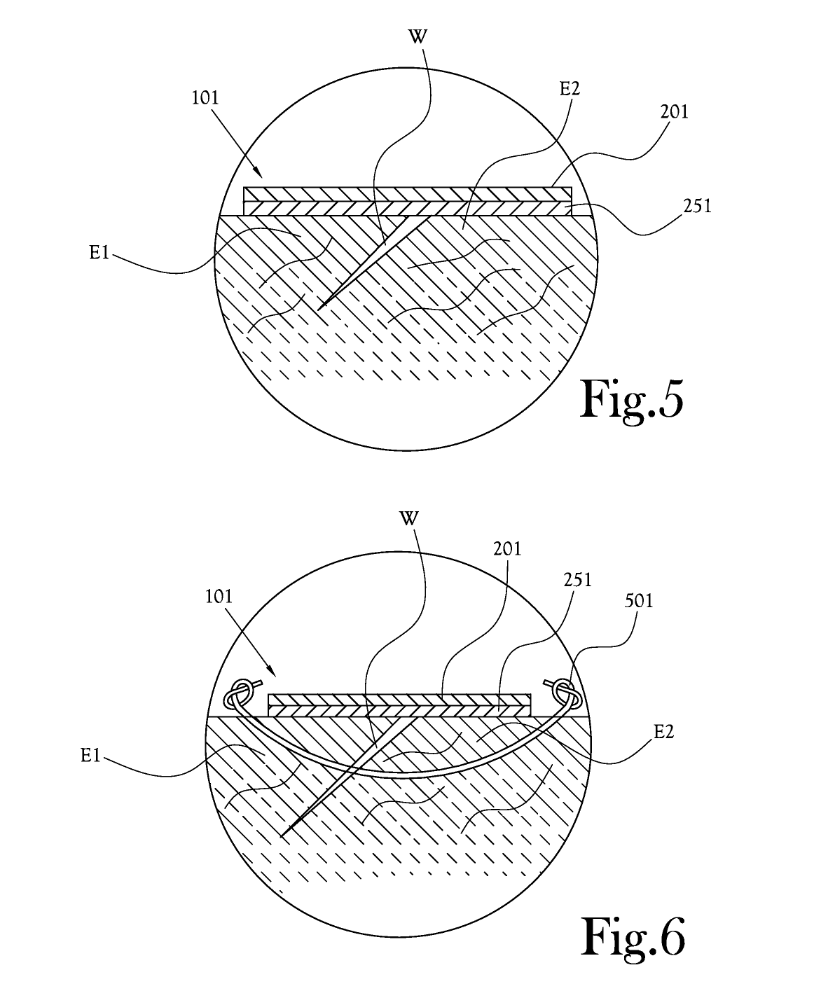

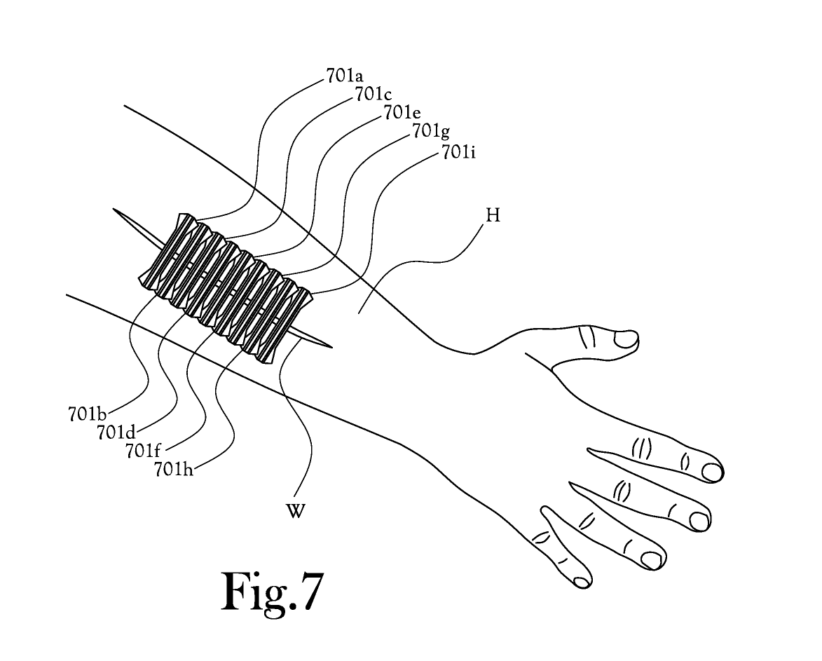

FIG. 4 shows one example embodiment of the present invention, in which a number of reinforced suture strips are employed to stabilize a wound on a human arm. As shown in FIG. 4, the suture strips 101a-f are positioned in a parallel series so that each suture strip 101a-f is roughly perpendicular to the line of the incisive wound W on the human body H. FIG. 5 shows a sectional view of one of the suture strips 101a positioned across the wound W, with one end of the suture strip 101a on either side of the wound W. The reinforced suture strip 101a is flexible and resilient enough to be applied on the skin and to give slightly, when necessary, so as to avoid breaking or tearing; at the same time, the semi-rigid suture strip 101a inhibits movement by the two areas of skin tissue E1 and E2 on opposing sides of the incisive wound W. In some embodiments, as shown in FIG. 6, a reinforced suture strip 101 is used in conjunction with a conventional suture 501.

FIG. 7 shows another example embodiment of the present invention, in which a number of overlapping reinforced suture strips are employed to stabilize a wound on a human arm. As shown in FIG. 7, the suture strips 701a-i are positioned in a parallel, overlapping series so that each suture strip 701a-i is roughly perpendicular to the line of the incisive wound W on the human body H. As shown, the end regions of each suture strip partially overlap or are partially overlapped by the end regions of one or more adjacent suture strips. Thus, the suture strips 701a-i form a connected assembly of suture strips. During use, the connectedness of the reinforced suture strips 701a-i enhances their stability relative to the incisive wound W and the moving skin of the patient; for each particular suture stip, one or more adjacent suture strips help to hold that particular suture strip in place. During removal of the suture strips 701a-i, the connectedness of the suture strips 701a-i facilitates the removal of the suture strips 701a-i, as the suture strips 701a-i effectively are removed as a single assembly.

In many embodiments, the reinforced suture strip inhibits or minimizes both abductive and transverse movement by the opposing sides of flesh about an incisive wound. By substantially immobilizing the two areas of skin tissue on opposing sides of an incisive wound, the reinforced suture strip helps to keep abutting skin tissue on opposing sides of the incisive wound from sliding or separating with respect to each other. By substantially immobilizing the two areas of skin tissue on opposing sides of an incisive wound, the reinforced suture strip helps to prevent suture stretching and helps to prevent any widening of an incision of reopening of a wound that has begin to close. Preventing reopening of the wound helps to avoid infection of the wound or the entry of foreign biological and non-biological entities. Further, by substantially immobilizing the skin tissue around an incisive wound, the reinforced suture strip facilitates the formation of linkages between areas of skin tissue on opposing sides of the incisive wound, hastening the healing process and helping to minimize the development of scar tissue and the appearance of scars.

FIGS. 5 and 6 illustrate an example of an angled incision or angled incisive wound, which is part of a method used for reducing the appearance of scars from incisions. In some circumstances, such as surgery, it is necessary for a medical practitioner to make an incision in the patient's skin. If the incision made is a vertical incision—i.e., the main direction of the incision is substantially perpendicular to the plane of the patient's top-most layer of skin—then, during the healing process, the areas of skin on opposing sides of the incision must form fibrous cross-linkages in a largely horizontal direction—that is, substantially parallel to the top-most layer of skin. Because the separated areas of skin on opposing sides of the incision will also likely be pulled and moved in a largely horizontal direction during the healing process, the body's healing process must work almost directly against the expected movement of the separated areas of skin. On the other hand, if the incision is made angled—that is, the angle of incision is substantially acute with respect to the plane of the top-most layer of skin adjacent the incision—then, during the healing process, the areas of skin on opposing sides of the incision will form fibrous cross-linkages in substantially angled and vertical directions, without directly competing with the horizontal movement of the separated opposing areas of skin on either side of the incision. FIGS. 5 and 6 show a reinforced suture strip enhancing and supplementing the beneficial aspects of the angled incision or angled incisive wound. By substantially immobilizing the two areas of skin tissue on opposing sides of the angled incisive wound, the reinforced suture strip helps to keep abutting skin tissue on opposing sides of the angled incisive wound from sliding or separating with respect to each other.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A method of treating an incision to minimize the development of scars about the incision, comprising:
providing an incision wherein the direction of the incision forms an acute angle with the plane of the top-most layer of a patient's skin adjacent the incision;
suturing the incision; and
applying a reinforced suture strip to the patient's skin about the incision, said reinforced suture strip including a length of material comprising a middle region and two end regions, said reinforced suture strip being applied to the patient's skin about the incision so that each end region is positioned on an opposing side of the incision from the other end region, said reinforced suture strip including:
a bottom layer to be applied directly against the patient's skin;
a top layer opposite to the bottom layer; and
at least two parallel band members positioned between the top layer and the bottom layer, each said band extending along the length of the material to restrict the movement of the adjacent skin relative to the incision.

2. The method of claim 1 wherein said bottom layer is fabricated from a non-woven, spunlaced polymer fabric.

3. The method of claim 1 wherein said top layer is fabricated from a non-woven, breathable polymer fabric.

4. The method of claim 1 wherein said band members are fabricated from a resilient, semi-rigid material.

5. The method of claim 1 wherein said band members of said reinforced suture strip are fabricated from a plastic material.

6. The method of claim 1 further comprising strips of adhesive interface material positioned between the band members and the bottom layer, said strips of adhesive interface material being fabricated from a pressure-sensitive acrylic adhesive material.

7. The method of claim 1 wherein said bottom layer is fabricated from a polyester fabric.

8. The method of claim 1 further comprising applying multiple reinforced suture strips to the patient's skin about the incision.

9. The method of claim 8 wherein at least two of said multiple reinforced suture strips partially overlap.

10. A method of treating an incision to minimize the development of scars about the incision, comprising:
providing an incision wherein the direction of the incision forms an acute angle with the plane of the top-most layer of a patient's skin adjacent the incision; and
applying a reinforced suture strip to the patient's skin about the incision, said reinforced suture strip including a length of material comprising a middle region and two end regions, said reinforced suture strip being applied to the patient's skin about the incision so that each end region is positioned on an opposing side of the incision from the other end region, said reinforced suture strip including:
a bottom layer to be applied directly against the patient's skin;
a top layer opposite to the bottom layer; and
at least two parallel band members positioned between the top layer and the bottom layer, each said band extending along the length of the material to restrict the movement of the adjacent skin relative to the incision.

11. The method of claim 10 wherein said bottom layer is fabricated from a non-woven, spunlaced polymer fabric.

12. The method of claim 10 wherein said top layer is fabricated from a non-woven, breathable polymer fabric.

13. The method of claim 10 wherein said band members are fabricated from a resilient, semi-rigid material.

14. The method of claim 10 wherein said band members of said reinforced suture strip are fabricated from a plastic material.

15. The method of claim 10 further comprising strips of adhesive interface material positioned between the band members and the bottom layer, said strips of adhesive interface material being fabricated from a pressure-sensitive acrylic adhesive material.

16. The method of claim 10 wherein said bottom layer is fabricated from a polyester fabric.

17. The method of claim 10 further comprising applying multiple reinforced suture strips to the patient's skin about the incision.

18. The method of claim 17 wherein at least two of said multiple reinforced suture strips partially overlap.

19. A reinforced suture strip for treating an incisive wound so as to minimize movement of skin around the incisive wound, comprising:
a length of material comprising a middle region and two end regions, said reinforced suture strip being applied to the patient's skin about the incision so that each end region is positioned on an opposing side of the incision from the other end region, said reinforced suture strip including:
a bottom layer to be applied directly against the patient's skin;
a top layer opposite to the bottom layer; and
at least two parallel band members positioned between the top layer and the bottom layer, each said band extending along the length of the material to restrict the movement of the adjacent skin relative to the incision,
wherein said reinforced suture strip, when applied to a patient's skin about the incision, each end region is positioned on an opposing side of the incision from the other end region to substantially immobilize each opposing side with respect to the incision, thereby keeping abutting skin tissue on opposing sides of the incisive wound from sliding or separating with respect to each other.

20. The reinforced suture strip of claim 19 further comprising strips of adhesive interface material positioned between the band members and the bottom layer, said strips of adhesive interface material being fabricated from a pressure-sensitive acrylic adhesive material.

21. The reinforced suture strip of claim 19 wherein said band members of said reinforced suture strip are fabricated from a plastic material.

22. The reinforced suture strip of claim 19 wherein said bottom layer is fabricated from a polyester fabric.

23. The reinforced suture strip of claim 19 wherein said bottom layer is fabricated from a non-woven, spunlaced polymer fabric, said top layer is fabricated from a non-woven, breathable polymer fabric, and said band members are fabricated from a resilient, semi-rigid material.
